(19) United States
(12) Patent Application Publication
(10) Pub. No.: US 2025/0391169 A1
(43) Pub. Date: Dec. 25, 2025

(54) AUGMENTED REALITY IMPROVEMENTS FOR HEALTHCARE

(71) Applicant: Welch Allyn, Inc., Skaneateles Falls, NY (US)

(72) Inventors: David E. Quinn, Moravia, NY (US); Terri Kapur, Sharon, MA (US)

(21) Appl. No.: 19/240,240

(22) Filed: Jun. 17, 2025

Related U.S. Application Data
(60) Provisional application No. 63/662,548, filed on Jun. 21, 2024.

(51) Int. Cl. G06V 20/40; G06V 10/70; G06V 10/82

(52) U.S. Cl. CPC G06V 20/44; G06V 10/82; G06V 20/41; G06V 10/768; G06V 20/47; G06V 2201/10

(57) ABSTRACT

A system provides augmented reality in a healthcare facility. The system receives a video feed of the healthcare facility. The video feed captured by a camera mounted on a device worn by a caregiver. The system identifies a patient environment in the healthcare facility based on the video feed, and detects an alarm triggered inside the patient environment. The system displays a second video feed of the patient environment on a head-up display on the device worn by the caregiver. The second video feed is captured contemporaneously with an event that triggered the alarm inside the patient environment.

### CROSS-REFERENCE TO RELATED APPLICATION(S)

This application claims the benefit of and priority to U.S. Provisional Patent Application No. 63/662,548, filed Jun. 21, 2024, the entire disclosure of which is incorporated by reference herein in its entirety.

### BACKGROUND

A quantity of patients that a nurse can effectively care for in a healthcare setting is an important consideration for ensuring quality patient care. Lower nurse-to-patient ratios are typically associated with reduced mortality rates and shorter hospital stays because lower nurse-to-patient ratios allow nurses to provide adequate attention and care to each patient.

However, many healthcare facilities experience shortages of qualified nurses, which can affect the quality of healthcare provided by the facilities because there are fewer nurses caring for more patients. Nursing shortages are primarily due to factors such as an aging population, increased healthcare needs, and lack of qualified nurse training. Nursing shortages are particularly acute in rural areas and underserved communities, further exacerbating healthcare disparities. Given the foregoing, it is desirable to develop tools that improve productivity and effectiveness of nurses to improve the quality patient care.

### SUMMARY

In general terms, the present disclosure relates to augmented reality in healthcare communications. In one possible configuration, a video feed of a patient environment is displayed on a head-up display worn by a caregiver, and the video feed is contemporaneous with an event that triggers an alarm inside the patient environment. Various aspects are described in this disclosure, which include, but are not limited to, the following aspects.

One aspect relates to a system for providing augmented reality in a healthcare facility, the system comprising: at least one processing device; and at least one computer readable data storage device storing software instructions that, when executed by the at least one processing device, cause the at least one processing device to: receive a video feed of the healthcare facility, the video feed captured by a camera mounted on a device worn by a caregiver; identify a patient environment in the healthcare facility based on the video feed; detect an alarm triggered inside the patient environment; and display a second video feed of the patient environment on a head-up display on the device worn by the caregiver, the second video feed captured contemporaneously with an event that triggered the alarm inside the patient environment.

Another aspect relates to a method of providing augmented reality in a healthcare facility, the method comprising: receiving a video feed of the healthcare facility, the video feed captured by a camera mounted on a device worn by a caregiver; identifying a patient environment in the healthcare facility based on the video feed; detecting an alarm triggered inside the patient environment; and displaying a second video feed of the patient environment on a head-up display on the device worn by the caregiver, the second video feed captured contemporaneously with an event that triggered the alarm inside the patient environment.

Another aspect relates to a device for providing augmented reality in a healthcare facility, the device comprising: a main housing; a lens attached to the main housing, the lens being made of a transparent material allowing the caregiver to see through the lens; a projector displaying data onto the lens; at least one processing device housed in the main housing; and at least one computer readable data storage device housed in the main housing, the at least one computer readable data storage device storing software instructions that, when executed by the at least one processing device, cause the at least one processing device to: display a video feed of a patient environment on the lens, the video feed captured contemporaneously with an event that triggered an alarm inside the patient environment.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 illustrates an example of a system that provides augmented reality for improving productivity and effectiveness of caregivers in a healthcare facility.

FIG. 2 schematically illustrates an example of an augmented reality server that facilitates the augmented reality in the healthcare facility of FIG. 1.

FIG. 3 illustrates an isometric view of an example of an augmented reality device worn by a caregiver in the healthcare facility of FIG. 1.

FIG. 4 illustrates an example of a head-up display on the augmented reality device of FIG. 3 from a point-of-view of the caregiver in the healthcare facility of FIG. 1.

FIG. 5 schematically illustrates an example of a method of providing augmented reality in the healthcare facility of FIG. 1.

FIG. 6 schematically illustrates another example of a method of providing augmented reality in the healthcare facility of FIG. 1.

### DETAILED DESCRIPTION

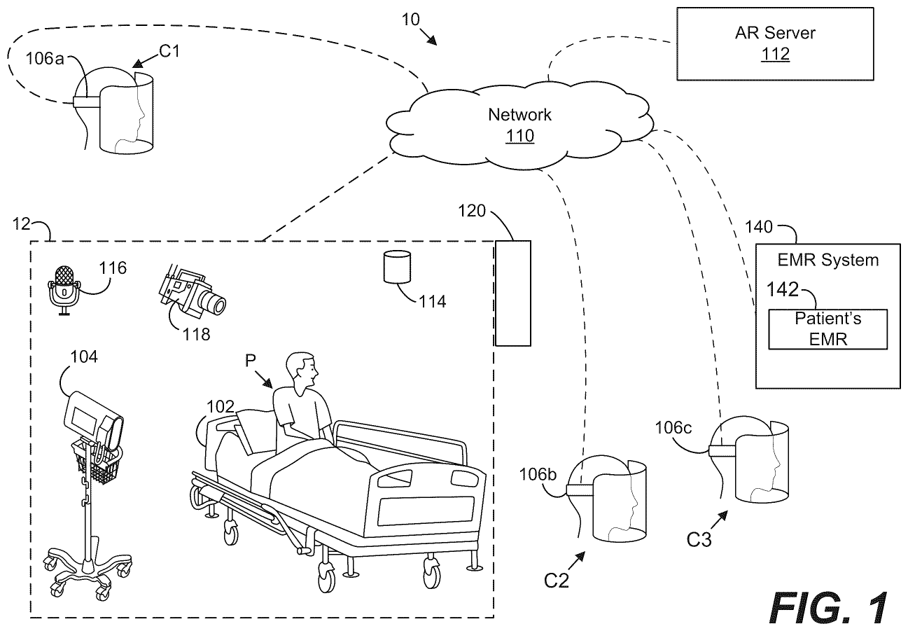

FIG. 1 illustrates an example of a system 10 that provides augmented reality for improving productivity and effectiveness of caregivers in a healthcare facility. Illustrative examples of the healthcare facility can include a hospital, a nursing home, a rehabilitation center, a long-term care facility, a medical office, and the like. The system 10 can share similar aspects with the system described in U.S. patent application Ser. No. 18/608,028, filed Mar. 18, 2024, the disclosure of which is herein incorporated by reference in its entirety.

In FIG. 1, a patient P is shown located inside a patient environment 12, which can be a room or other designated area within the healthcare facility. For example, the patient environment 12 can include a patient room within a hospital. As shown in FIG. 1, caregivers C are located outside of the patient environment 12 in other areas of the healthcare facility.

In the example illustrated in FIG. 1, a plurality of caregivers (e.g., a first caregiver C1, a second caregiver C2, and a third caregiver C3) are present in the healthcare facility. The first, second, and third caregivers C1, C2, C3 that are illustrated in FIG. 1 are provided by way of illustrative example, and it is contemplated that the number of caregivers in the healthcare facility can be significantly greater than the three caregivers shown in FIG. 1.

The patient P is shown supported on a patient support apparatus 102 inside the patient environment 12. In some examples, the patient support apparatus 102 is a hospital bed, or similar type of apparatus. The patient support apparatus 102 can include one or more sensors that measure one or more physiological parameters of the patient P. For example, the one or more sensors can measure one or more vital signs such as heart rate and respiration rate. In further examples, the one or more sensors can measure patient weight, patient motion, and patient activity level. In further examples, the one or more sensors can detect patient exit.

The patient support apparatus 102 includes an alarm system that generates alarms based on data obtained from the one or more sensors included on the patient support apparatus. As an example, the alarm system can trigger an alarm when the one or more sensors detect the patient P is about to exit the patient support apparatus 102 and a caregiver C is not present inside the patient environment 12. As a further illustrative example, the alarm system of the patient support apparatus 102 can trigger an alarm when the one or more sensors detect changes in one or more vital signs such as respiration rate and/or heart rate that indicate health deterioration. Additional types of alarms triggered by the alarm system of the patient support apparatus 102 are possible. The alarms generated by the patient support apparatus 102 are transmitted from the patient environment 12 to an augmented reality server 112 via a network 110.

As further shown in FIG. 1, a monitoring device 104 is monitoring the patient P. The monitoring device 104 is positioned next to the patient support apparatus 102. The monitoring device 104 includes one or more sensors that can measure physiological parameters of the patient such as blood oxygen saturation, body temperature, blood pressure, pulse/heart rate, respiration rate, electrocardiogram (EKG), and the like. In some examples, the monitoring device 104 is a spot monitor that monitors a health status of the patient P either continuously or episodically.

The monitoring device 104 can also include an alarm system that generates alarms based on data obtained from the one or more sensors included on the monitoring device 104. As an illustrative example, when an individual physiological parameter falls outside of a predetermined range, the alarm system of the monitoring device 104 triggers an alarm. As another illustrative example, the monitoring device 104 can compute an early warning score based on a combination of the physiological parameters. When the early warning score exceeds a predetermined threshold, the alarm system of the monitoring device 104 triggers an alarm requesting immediate intervention by the caregivers C in the healthcare facility. The alarms generated on or by the monitoring device 104 can be transmitted from the patient environment 12 to the augmented reality server 112 via the network 110.

The patient support apparatus 102 and the monitoring device 104 are connected to the network 110. When an alarm is triggered by data collected from any one of these devices in the patient environment 12, the alarm is transmitted to the augmented reality server 112 via the network 110. The patient environment 12 can include additional devices to monitor a status of the patient P. These additional types of devices can trigger additional types of alarms that can be communicated to the augmented reality server 112 for intervention by the caregivers C.

As shown in FIG. 1, the patient environment 12 can further include an antenna 114 that emits a wireless signal detectable by augmented reality devices 106 that are worn or otherwise carried by the caregivers C. For example, the antenna 114 can emit a radio frequency (RF) signal, an optical (e.g., infrared) signal, an acoustic (e.g., ultrasound) signal, a Bluetooth signal, or other type of wireless signal detectable by the augmented reality devices 106. As will be described in more detail below, the signal emitted by the antenna 114 can be used to identify the patient environment 12 among a plurality of patient environments in the healthcare facility.

The patient environment 12 can include a signage 120 that is mounted outside of the patient environment 12. The signage 120 can include identifiers such as a room number that identifies the patient environment 12 (e.g., Room 212). Additionally, or alternatively, the signage 120 can include a machine-readable label such as a quick-response (QR) code that can be scanned by a camera on an augmented reality device 106 to identify the patient environment 12.

The system 10 can include a microphone 116 that captures sounds inside the patient environment. The microphone 116 transmits the sounds of the patient environment 12 to the augmented reality server 112 via the network 110. In some examples, the augmented reality server 112 analyzes the sounds captured by the microphone 116 to determine a status of the patient P such as whether the patient P needs an intervention, or not.

The system 10 can include a camera 118 that captures images of the patient environment 12. The camera 118 transmits the images of the patient environment 12 to the augmented reality server 112 via the network 110. In some examples, the augmented reality server 112 analyzes the images captured by the camera 118 to determine a status of the patient P.

In further examples, the augmented reality server 112 analyzes both the images captured by the camera 118 and the sounds captured by the microphone 116 to determine a status of the patient P. In such examples, the augmented reality server 112 generates alarms requesting immediate intervention by the caregivers C based on a status of the patient P determined from both the images and sounds captured inside the patient environment 12.

The system 10 can include an electronic medical record (EMR) (also called electronic health record (EHR)) system 140. The EMR system 140 manages the patient P's medical history and information. The EMR system 140 can be operated by a healthcare service provider, such as a hospital or medical clinic. The EMR system 140 stores an EMR 142 of the patient P.

The augmented reality server 112 can communicate with the EMR system 140 via the network 110. In some examples, the augmented reality server 112 determines a status of the patient P based on data stored in the EMR 142 of the patient P.

In the example shown in FIG. 1, the augmented reality devices 106 include medical visors or face shields worn by the caregivers C. The augmented reality devices 106 can include alternative form factors such as eyeglasses or other headgear, headwear, and articles that can be worn by caregivers such that the disclosure provided herein is not limited to medical visors.

The augmented reality devices 106 include both mechanical and electronic components that enhance daily tasks and workflows performed by the caregivers C, and which can result in improved patient outcomes. The augmented reality devices 106 each include a head-up display and 3D audio to provide an augmented reality interactive experience with sensorial feedback. The augmented reality devices 106 can be controlled by the augmented reality server 112 to provide the augmented reality that provides enhanced information to the caregivers C.

The augmented reality server 112 can provide the augmented reality on the augmented reality devices 106 worn by the caregivers C based on the alarms triggered inside the patient environment 12 and/or the status of the patient P. The augmented reality can include providing a video and/or audio feed of the patient P captured immediately prior, during, and immediately after an alarm is triggered inside the patient environment 12. In some instances, the augmented reality enables the caregiver C to control one or more devices inside the patient environment 12 when the caregiver C is not physically located inside the patient environment 12.

As an illustrative example, the augmented reality can allow the caregiver C to control the patient support apparatus 102 and/or the monitoring device 104 to suppress an alarm triggered on these devices when the video and/or audio feed provided by the augmented reality indicates that the alarm is a false alarm or that the status of the patient P is safe. The augmented reality can improve the effectiveness and efficiency of the caregiver C because the caregiver C does not need to physically enter the patient environment 12 to suppress a false alarm.

As an illustrative example, when the video and/or audio feed provided by the augmented reality indicates that the alarm is due to an adverse event or condition in the patient environment 12, the augmented reality allows the caregiver C to address the adverse event or condition either by remotely controlling one or more devices inside the patient environment 12, or by physically entering the patient environment 12 to provide in-patient case. Accordingly, the augmented reality can reduce response times to adverse events, and thereby improve patient care. Further, the augmented reality enables the caregiver C to determine whether there is a trend that led to the alarm being triggered such that corrective actions can be taken to mitigate the trend.

The network 110 facilitates data communication between the devices inside the patient environment 12, including between the patient support apparatus 102, the monitoring device 104, the antenna 114, the microphone 116, and the camera 118. The network 110 can further facilitate communication between the devices inside the patient environment 12 and systems that are located outside of the patient environment 12 such as the augmented reality server 112 and the EMR system 140. Also, the network 110 facilitates data communication between the augmented reality devices 106 that are worn or carried by the caregivers C.

The network 110 can include routers and other networking devices. The network 110 can include any type of wired or wireless connection, or any combinations thereof. Wireless connections in the network 110 can include Bluetooth, Wi-Fi, Zigbee, cellular network connections such as 4G or 5G, and other similar types of wireless technologies.

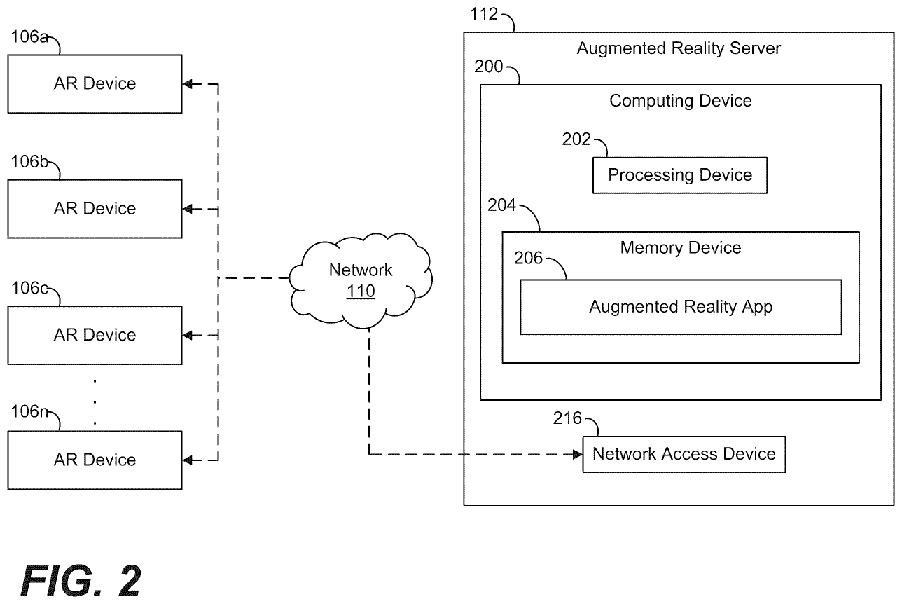

FIG. 2 schematically illustrates an example of the augmented reality server 112 that facilitates the augmented reality in the healthcare facility. As shown in FIG. 2, the augmented reality server 112 is in communication over the network 110 with the augmented reality devices 106 worn by the caregivers C in the healthcare facility.

The augmented reality server 112 includes a computing device 200 having a processing device 202. The processing device 202 is an example of a processing unit such as a central processing unit (CPU). The processing device 202 can include one or more CPUs. The processing device 202 can include electronic computing devices and/or circuits such as digital signal processors, field-programmable gate arrays, integrated circuits, and similar devices.

The augmented reality server 112 includes a memory device 204 that stores data and instructions for execution by the processing device 202. As shown in FIG. 2, the memory device 204 stores an augmented reality application 206, which is described in more detail below. The memory device 204 includes computer readable media, including computer readable media accessible by the processing device 202. For example, computer readable media includes computer readable storage media and computer readable communication media.

Computer readable storage media includes volatile and nonvolatile, removable, and non-removable media implemented in any device configured to store information such as computer readable instructions, data structures, program modules, or other data. Computer readable storage media can include random access memory, read only memory, electrically erasable programmable read only memory, flash memory, and other memory technology, including any medium that can be used to store information that can be accessed by the augmented reality server 112. The computer readable storage media is non-transitory.

Computer readable communication media embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are within the scope of computer readable media.

The augmented reality server 112 further includes a network access device 216. The network access device 216 operates to communicate with other computing devices over the network 110 such as the augmented reality devices 106 worn by the caregivers C. Examples of the network access device 216 include wired network interfaces and wireless network interfaces.

The augmented reality application 206 shares data across devices and sub-systems of the system 10. For example, the augmented reality application 206 synchronizes data captured by the devices inside the patient environment 12, such as the patient support apparatus 102 and the monitoring device 104, for display on the head-up displays of the augmented reality devices 106. The augmented reality application 206 can further playback on the augmented reality devices 106 the audio and images captured from the microphone 116 and the camera 118, respectively.

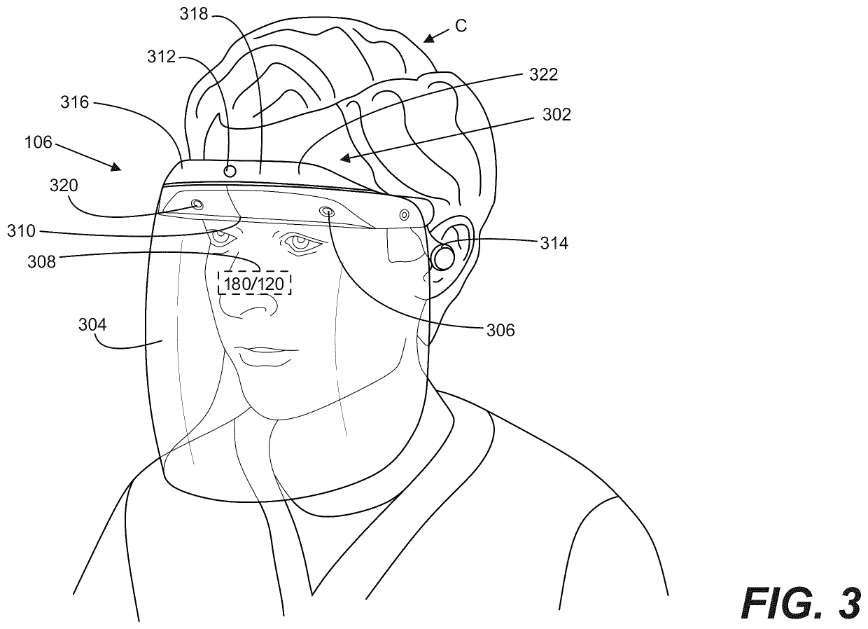

FIG. 3 illustrates an isometric view of an example of the augmented reality device 106 worn by a caregiver C in the healthcare facility. The augmented reality device 106 is a hardware platform that is based on personal protective equipment (PPE) used in healthcare settings. The hardware platform is connected to the EMR system 140, one or more sensors collecting data from the patient P inside the patient environment 12, and other devices. The hardware platform can facilitate prompt, prioritized patient care, reduction in alarm fatigue, improved communication between caregivers and patients, and richer and passively collected documentation of patient condition and clinician-patient interactions.

In the example of FIG. 3, the hardware platform is enabled with a replaceable visor or face shield. The visor or face shield protects the caregiver C from air-borne liquid droplets and particles that can potentially have viruses and bacteria, while allowing other persons to see the face of the caregiver C. For example, the face shield surrounds the face of the caregiver C. The augmented reality devices 106 can include alternative form factors such as eyeglasses or other headgear, headwear, and articles that can be worn by caregivers such that the disclosure provided herein is not limited to medical visor or face shield embodiment shown in FIG. 3.

The augmented reality device 106 includes a main housing 302 that houses electronic components for providing an augmented reality interactive experience. The main housing 302 includes a power source 318 such as one or more rechargeable batteries. The main housing further includes a computing device 322 that can include similar aspects as the computing device 200 of the augmented reality server 112, which is described above with reference to FIG. 2.

In the example shown in FIG. 3, the augmented reality device 106 includes a lens 304 removably attached to the main housing 302. The lens 304 is made of a transparent material allowing the caregiver C to see through the lens. In some examples, the lens 304 is a prescriptive lens that corrects myopia (i.e., nearsightedness), hyperopia (i.e., farsightedness), or other conditions that may affect the visual acuity of the caregiver C.

In the example shown in FIG. 3, the lens 304 has a form factor of a visor. For example, the lens 304 is shaped and sized to surround the face of the caregiver C to protect the caregiver C from air-borne liquid droplets and particles. In examples where the augmented reality device has the form factor of eyeglasses, the lens can have a different shape and size.

In examples where the lens 304 has a form factor of a visor, the main housing 302 can be reusable, while the lens 304 can be a disposable component that is replaced and discarded after one or more uses. In some examples, the lens 304 is made from a recyclable plastic.

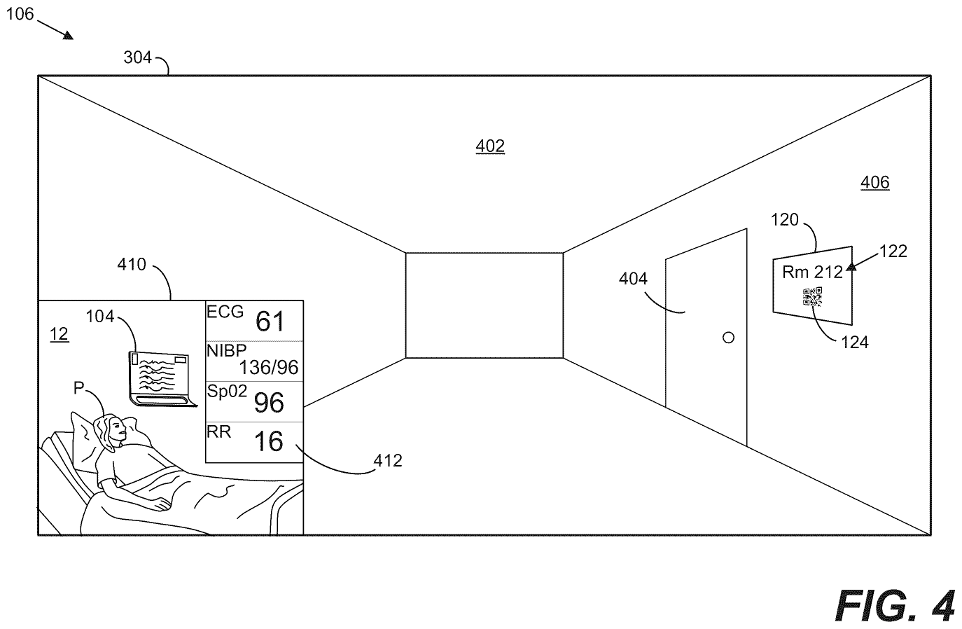

The augmented reality device 106 includes a projector 306 that projects a data 308 onto the lens 304. In some examples, the projector 306 is a Pico projector. The data 308 that is projected onto the lens 304 transforms the lens 304 into a head-up display that provides augmented reality visuals for the caregiver C. For example, the lens 304 allows the caregiver C can see their surroundings with the data 308 superimposed thereon. An illustrative example of the head-up display is shown in FIG. 4, which will be described in more detail.

In some instances, the data 308 displayed on the lens 304 can be pulled from the EMR 142 of the patient P stored on the EMR system 140. Alternatively, the data 308 displayed on the lens 304 can be pulled directly from devices inside the patient environment 12 such as the patient support apparatus 102, the monitoring device 104, and the camera 118.

The data 308 displayed on the lens 304 can include a video captured by the camera 118 inside the patient environment 12. The video displayed in the data 308 can include a previously captured video such as a video captured by the camera 118 before, during, and after an event is detected inside the patient environment 12 such as an event that triggers an alarm inside the patient environment 12. In such examples, the video displayed on the lens 304 is contemporaneous with the event that triggered the alarm inside the patient environment 12. Alternatively, the video displayed in the data 308 can include a real-time or a near real-time video feed of the patient environment 12 captured by the camera 118.

As further shown in FIG. 3, the augmented reality device 106 includes a microphone 310 that records audio from the caregiver C as well as the surroundings of the caregiver C. For example, the microphone 310 can record a verbal utterance from the caregiver C that acts as an input to remotely control one or more devices inside the patient environment 12 when the caregiver C is physically located outside of the patient environment 12. The microphone 310 can also record conversations between the caregiver C, the patient P, and other caregivers. The microphone 310 can enable two-way communications between the caregiver C and the patient P when used in combination with the microphone 116 positioned inside the patient environment 12. The two-way communications between the caregiver C and the patient P can help the caregiver C to remotely evaluate a condition inside the patient environment 12 such as alarms triggered by one or more devices inside the patient environment 12.

The augmented reality device 106 includes a camera 312 that captures images from a point-of-view of the caregiver C. The camera 312 can capture images of the healthcare facility from the point-of-view of the caregiver C. patient P while being cared for by the caregiver C. The camera 312 can also record images of user interfaces of the patient support apparatus 102, the monitoring device 104, and other devices/sensors while being used by the caregiver C. The camera 312 can also capture images of the patient environment 12. For example, the camera 312 can be used to capture a video of the patient P for entry into the EMR 142 of the patient P along with a level of consciousness determination. The captured video provides objective evidence of the patient's behavior, such as whether the caregiver considers the patient to be groggy or agitated, which can facilitate a handoff to the next nurse on duty.

The augmented reality device 106 includes a headphone 314. The headphone can provide 3D audio to further enhance the augmented reality interactive experience provided on the augmented reality device 106. The headphone 314 can include a bone conduction speaker.

As further shown in FIG. 3, the augmented reality device 106 can include a haptic actuator 316 that provides haptic feedback such as vibrations based on the alerts received from the augmented reality server 112. For example, the haptic actuator 316 can vibrate when an alarm is received from the patient environment 12. The haptic feedback from the haptic actuator 316 can alert the caregiver C about an alarm event triggered inside the patient environment 12. Additionally, the haptic actuator 316 can provide haptic feedback such as a vibration when new data relevant to the condition of the patient P is available from any one of the devices located in the patient environment 12 and/or the EMR 142 of the patient P.

The augmented reality device 106 can further include one or more sensors that detect inputs from the caregiver C such as hand gestures, eye movements, and verbal utterances. In some examples, the hand gestures are detected by the camera 312 that also captures the video of the healthcare facility from the point-of-view of the caregiver C. In some examples, the one or more sensors can include a secondary camera 320 that is orientated toward the caregiver C's face for tracking the eye movements of the caregiver C. In some further examples, the verbal utterance can be detected by the microphone 310 of the augmented reality device 106.

The hand gestures, eye movements, and verbal utterances from the caregiver C detected by the augmented reality device 106 can be used as inputs to control the data 308 displayed on the lens 304. Further, the hand gestures, eye movements, and verbal utterances detected by the augmented reality device 106 can be used as inputs to remotely control one or more devices inside the patient environment 12 such as the patient support apparatus 102, the monitoring device 104, and other devices. In some examples, the hand gestures, eye movements, and verbal utterances detected by the augmented reality device 106 can be used as inputs to remotely suppress an alarm triggered inside the patient environment 12.

FIG. 4 illustrates an example of a head-up display 402 on the augmented reality device 106 from a point-of-view of a caregiver who is wearing the augmented reality device 106. As described above, the head-up display 402 is projected onto the lens 304 of the augmented reality device 106. In this illustrative example, the head-up display 402 shows a view of a hallway inside the healthcare facility that includes a door 404 to the patient environment 12. In this illustrative example, the caregiver C is physically located outside of the patient environment 12. The door 404 is closed and the hallway includes a wall 406 such that the caregiver C who is wearing the augmented reality device 106 cannot see inside the patient environment 12.

In the example shown in FIG. 4, the signage 120 is mounted on the wall 406 outside of the patient environment 12. The signage 120 includes information that identifies the patient environment 12 such as a room number 122 or a machine-readable label 124 such as a QR code.

In some examples, the camera 312 of the augmented reality device 106 captures a video feed in which the signage 120 is visible, and the augmented reality device 106 transmits the video feed to the augmented reality server 112. In such examples, the computing device 200 of the augmented reality server 112 can identify the patient environment 12 based on the information in the signage 120 such as the room number 122 or the machine-readable label 124. Alternatively, the computing device housed on the augmented reality device 106 can process the video feed captured by the camera 312 to identify the patient environment 12 based on the information in the signage 120 such as the room number 122 or the machine-readable label 124.

When the patient environment 12 is identified, the projector 306 is controlled either by the computing device of the augmented reality device 106 or the computing device 200 of the augmented reality server 112 to display on the lens 304 a video feed 410 of the patient P captured by the camera 118 inside the patient environment 12. The video feed 410 can include a previously captured video such as a video captured by the camera 118 before, during, and after an event is detected inside the patient environment 12 such as when an alarm is triggered inside the patient environment 12. Alternatively, the video feed 410 can include a real-time or near real-time video feed of the patient environment 12 captured by the camera 118. Thus, the augmented reality provided on the augmented reality device 106 allows the caregiver to see inside the patient environment 12 when the caregiver is not physically inside the patient environment 12.

As further shown in FIG. 4, a parameter display 412 is overlayed on the video feed 410. The parameter display 412 includes one or more physiological parameters such as EKG measurements, non-invasive blood pressure, blood oxygen saturation (SpO2), respiration rate, pulse, and other parameters relevant to the health of the patient P. The one or more physiological parameters can be pulled from the EMR 142 of the patient P, or can be pulled directly from one or more devices monitoring the patient P inside the patient environment such as the patient support apparatus 102 and/or the monitoring device 104.

The one or more physiological parameters displayed in the parameter display 412 are synchronized with the video feed 410. For example, the one or more physiological parameters are contemporaneous with the video feed 410. By aligning the one or more physiological parameters in the parameter display 412 with the video feed 410, the augmented reality device 106 provides enhanced insight and information about the patient P which allows viewing the patient P with overlayed vitals and other health information at anytime from anywhere in the healthcare facility. Such information can prepare the caregiver C for providing in-person care to the patient P before the caregiver C enters the patient environment 12 such that the augmented reality device 106 provides a seamless transition between remote care and in-person care. Further, the augmented reality device 106 enhances situational awareness for the caregiver C, which can help to improve patient care, and potentially prevent future adverse events.

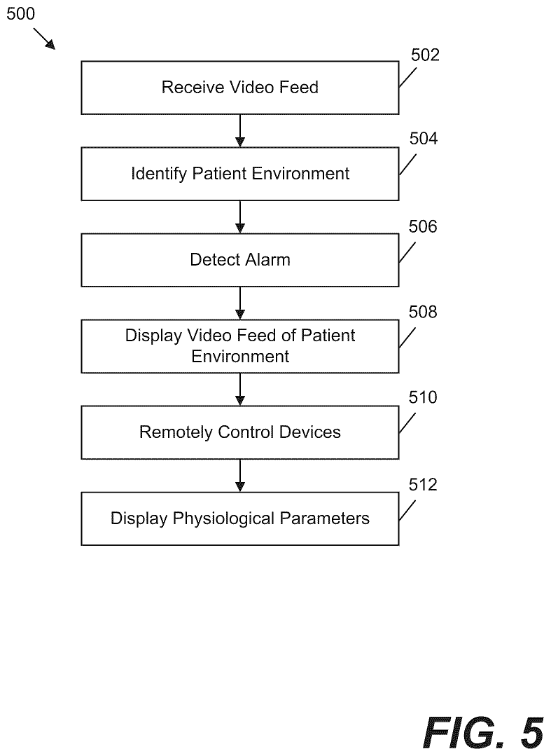

FIG. 5 schematically illustrates an example of a method 500 of providing augmented reality in the healthcare facility. More specifically, the method 500 provides augmented reality on an augmented reality device 106 worn by a caregiver C. In some examples, the method 500 can be performed by the augmented reality server 112. In alternative examples, the method 500 can be performed by the computing device 322 of the augmented reality device 106.

The method 500 includes an operation 502 of receiving a video feed of the healthcare facility. The video feed can be captured by the camera 312 mounted on the augmented reality device 106 worn by a caregiver C inside the healthcare facility. In such examples, the video feed that is received in operation 502 is from the point-of-view of the caregiver C.

The method 500 includes an operation 504 of identifying the patient environment 12. In some examples, the patient environment 12 is identified based on the video feed received in operation 502. For example, operation 504 can include identifying the patient environment 12 based on the signage 120 mounted outside of the patient environment 12. As described above, the signage 120 can include a room number 122 and/or a machine-readable label 124 that identifies the patient environment 12. In alternative examples, operation 504 includes identifying the patient environment 12 based on the wireless signal emitted from the antenna 114.

The method 500 includes an operation 506 of detecting an alarm triggered inside the patient environment 12. As described above, the alarm can be triggered by an event detected by a device inside the patient environment 12 such as the patient support apparatus 102, the monitoring device 104, or another device. The alarm can be transmitted via the network 110 from the device in the patient environment 12 to the augmented reality server 112. While FIG. 5 shows operation 506 as occurring after operations 502, 504, the order of operations may vary such that the operation 506 of detecting the alarm triggered inside the patient environment 12 can occur before the operation 502 of receiving the video feed of the healthcare facility.

The method 500 includes an operation 508 of displaying the video feed 410 of the patient P captured by the camera 118 inside the patient environment 12 on the head-up display 402 of the augmented reality device 106 worn by a caregiver C. The video feed 410 is captured contemporaneously with the event that triggered the alarm inside the patient environment 12.

In some examples, the method 500 can further include an operation 510 of remotely controlling one or more devices inside the patient environment 12 based on inputs detected from the caregiver C outside of the patient environment 12. The inputs can include at least one of a hand gesture, an eye movement, and a verbal utterance by the caregiver C. The inputs can be used to remotely control one or more devices inside the patient environment 12 such as the patient support apparatus 102, the monitoring device 104, and other devices. In some examples, the inputs are used to suppress the alarm triggered inside the patient environment 12.

In some examples, the method 500 can further include an operation 512 of displaying one or more physiological parameters captured contemporaneously with the event that triggered the alarm inside the patient environment 12. For example, the one or more physiological parameters can be displayed in the parameter display 412 that is overlayed on the video feed 410 of the patient P inside the patient environment 12, as shown in the example of FIG. 4.

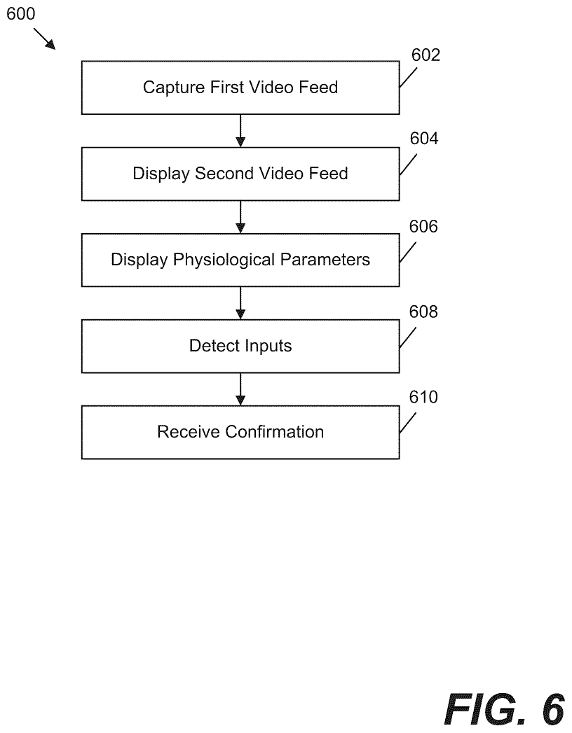

FIG. 6 schematically illustrates an example of a method 600 of providing augmented reality in the healthcare facility. The method 600 allows a caregiver to virtually visit and assess the patient P and address an alarm or any other situation detected in the patient environment 12 without physically entering the patient environment 12. The method 600 can be performed by the augmented reality device 106 when worn on the caregiver C as shown in FIG. 3.

The method 600 includes an operation 602 of capturing a first video feed of healthcare facility. Operation 602 can be performed using the camera 312 which captures the first video feed of healthcare facility from the point-of-view of the caregiver C.

The method 600 includes an operation 604 of displaying a displaying a second video feed of the patient environment 12 on the lens 304. The second video displayed in operation 604 is based on identification of the patient environment 12 from the first video feed captured in operation 602. In some examples, the second video feed is contemporaneous with an event that triggers an alarm inside the patient environment 12. In alternative examples, the second video feed is in real-time or a near real-time. An example of the second video feed displayed in operation 604 is the video feed 410 shown in FIG. 4.

The method 600 can further include an operation 606 of displaying one or more physiological parameters captured contemporaneously with the event that triggered the alarm inside the patient environment 12. In operation 606, the one or more physiological parameters can be overlayed on the second video feed of the patient environment 12, as shown in FIG. 4.

The method 600 can further include an operation 608 of detecting inputs for remotely controlling one or more devices inside the patient environment 12. The inputs can include at least one of a hand gesture, an eye movement, and a verbal utterance. The inputs can be detected by the microphone 310, the camera 312, and/or the secondary camera 320. The inputs detected in operation 608 can be used to remotely control one or more devices inside the patient environment such as to suppress an alarm triggered inside the patient environment 12. For example, the inputs detected in operation 608 can be used to remotely control the patient support apparatus 102, the monitoring device 104, or another device inside the patient environment 12.

The method 600 can further include an operation 610 of receiving a confirmation that control of the one or more devices inside the patient environment 12 is implemented based on the inputs detected in operation 608. For example, operation 610 can include providing haptic feedback such as vibrations using the haptic actuator 316 on the augmented reality device 106. As an illustrative example, the haptic feedback can provide a confirmation that an alarm has been suppressed based on the inputs detected in operation 608.

The various embodiments described above are provided by way of illustration only and should not be construed to be limiting in any way. Various modifications can be made to the embodiments described above without departing from the true spirit and scope of the disclosure.

What is claimed is:

1. A system for providing augmented reality in a healthcare facility, the system comprising:
at least one processing device; and
at least one computer readable data storage device storing software instructions that, when executed by the at least one processing device, cause the at least one processing device to:
receive a video feed of the healthcare facility, the video feed captured by a camera mounted on a device worn by a caregiver;
identify a patient environment in the healthcare facility based on the video feed;
detect an alarm triggered inside the patient environment; and
display a second video feed of the patient environment on a head-up display on the device worn by the caregiver, the second video feed captured contemporaneously with an event that triggered the alarm inside the patient environment.

2. The system of claim 1, wherein the patient environment is identified based on a signage mounted outside of the patient environment.

3. The system of claim 2, wherein the signage includes a room number or a machine-readable label that identifies the patient environment.

4. The system of claim 1, wherein the instructions, when executed by the at least one processing device, further cause the at least one processing device to:
remotely control one or more devices inside the patient environment based on inputs detected from the caregiver outside of the patient environment.

5. The system of claim 4, wherein the inputs include at least one of a hand gesture, an eye movement, and a verbal utterance.

6. The system of claim 4, wherein the inputs remotely control the one or more devices inside the patient environment to suppress the alarm triggered inside the patient environment.

7. The system of claim 1, wherein the instructions, when executed by the at least one processing device, further cause the at least one processing device to:
display one or more physiological parameters captured contemporaneously with the event that triggered the alarm inside the patient environment.

8. The system of claim 7, wherein the one or more physiological parameters are overlayed on the second video feed of the patient environment.

9. A method of providing augmented reality in a healthcare facility, the method comprising:
receiving a video feed of the healthcare facility, the video feed captured by a camera mounted on a device worn by a caregiver;
identifying a patient environment in the healthcare facility based on the video feed;
detecting an alarm triggered inside the patient environment; and
displaying a second video feed of the patient environment on a head-up display on the device worn by the caregiver, the second video feed captured contemporaneously with an event that triggered the alarm inside the patient environment.

10. The method of claim 9, further comprising:
remotely controlling one or more devices inside the patient environment based on inputs detected from the caregiver outside of the patient environment.

11. The method of claim 10, wherein the inputs remotely control the one or more devices inside the patient environment to suppress the alarm triggered inside the patient environment.

12. The method of claim 9, further comprising:
displaying one or more physiological parameters captured contemporaneously with the event that triggered the alarm inside the patient environment.

13. The method of claim 12, wherein the one or more physiological parameters are overlayed on the second video feed of the patient environment.

14. The method of claim 9, wherein identifying the patient environment is based on a room number or a machine-readable label in the video feed of the healthcare facility.

15. A device for providing augmented reality in a healthcare facility, the device comprising:
a main housing;
a lens attached to the main housing, the lens being made of a transparent material allowing the caregiver to see through the lens;
a projector displaying data onto the lens;
at least one processing device housed in the main housing; and
at least one computer readable data storage device housed in the main housing, the at least one computer readable data storage device storing software instructions that, when executed by the at least one processing device, cause the at least one processing device to:
display a video feed of a patient environment on the lens, the video feed captured contemporaneously with an event that triggered an alarm inside the patient environment.

16. The device of claim 15, wherein the instructions, when executed by the at least one processing device, further cause the at least one processing device to:
display one or more physiological parameters captured contemporaneously with the event that triggered the alarm inside the patient environment.

17. The device of claim 16, wherein the one or more physiological parameters are overlayed on the second video feed of the patient environment.

18. The device of claim 15, wherein the instructions, when executed by the at least one processing device, further cause the at least one processing device to:
detect inputs for remotely controlling one or more devices inside the patient environment, the inputs including at least one of a hand gesture, an eye movement, and a verbal utterance.

19. The device of claim 18, wherein the inputs remotely control the one or more devices inside the patient environment to suppress the alarm triggered inside the patient environment.

20. The device of claim 15, further comprising:
a camera mounted on the main housing, the camera configured to capture a video of the healthcare facility from a point of view of a person wearing the device; and
wherein the patient environment is identified based on a room number or a machine-readable label in the video of the healthcare facility captured by the camera.
